(19) United States
(12) Patent Application Publication
(10) Pub. No.: US 2007/0154887 A1
(43) Pub. Date: Jul. 5, 2007

(54) Method for the identification of syntenic regions

(75) Inventors: Luis Mendoza, Geneva (CH); MICHAEL DENNIS PRICKETT, YORK (GB)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: APPLIED RESEARCH SYSTEMS ARS HOLDING N.V., CURACAO (NL)

(21) Appl. No.: 10/547,866

(22) PCT Filed: Mar. 3, 2004
(86) PCT No.: PCT/EP04/50248

§ 371 Date: Sep. 26, 2006

(30) Foreign Application Priority Data
Mar. 4, 2003 (EP) 03100532.5
Sep. 24, 2003 (EP) 03103535.5

(51) Int. Cl. C12Q 1/68; G06F 19/00

(52) U.S. Cl. 435/6; 702/20

(57) ABSTRACT

The identification of the syntenic regions of a given genomic fragment conventionally involves a similarity-based search, and then taking the best hits and extending them manually until the whole region of interest is covered. Such a process is labor intensive and not suitable for a pipeline with thousands of sequences to analyze. The present invention consists of a method for the automatic identification of syntenic regions of a given input sequence, and its optimization to yield results with high specificity.

### FIELD OF THE INVENTION

This invention relates to a method and a computer program for the automated identification of genomic syntenic regions.

### BACKGROUND OF THE INVENTION

The availability of closely related genomes makes it possible to carry out genome-wise comparisons and analyses of synteny. Generally, “synteny” can be defined as the conservation of gene order (at least two genes) between genomic sequences in different species, regardless of the distance between the genes in the chromosome. Similarly, synteny can also be defined as two or more genes found together on a single chromosome in species A, which are also found together on a single chromosome in species B. A typical use of the term is: “Starting from a common ancestral genome approximately 75 Myr, the mouse and human genomes have each been shuffled by chromosomal rearrangements. The rate of these changes, however, is low enough that local gene order remains largely intact. It is thus possible to recognize syntenic (literally ‘same thread’) regions in the two species that have descended relatively intact from the common ancestor. (“Mouse Genome Sequencing Consortium (2002). Initial sequencing and comparative analysis of the mouse genome. Nature 420:520”).

The order of many genes, gene numbers, gene positions and even gene structures (exon-intron organization, splice site usage, and so on) remain highly conserved when two genomes have only recently diverged. New genes can be identified from direct genome comparisons. By comparing the genomes of several closely related species, conserved regulatory regions can also be easily identified (Bafna et al., The Conserved exon method for gene finding. Proc. Int. Conf. Intell. Syst. Mol. Biol. 2000, 8:3-12). For these reasons, making use of comparative genomic data is a key challenge for the gene-prediction field (Zang M. Q., Nature Genetics September 2002, 3:698-710; Pennacchio L. A. and Rubin E. M., Nature Genetics February 2001, 2:100-109).

The value of comparative genomics is illustrated by the sequencing of the mouse genome to achieve annotation of the human genome. In a traditional way, comparative genomics is based on large human-mouse sequence comparison. One interesting study concerned the ˜3.3 Mb of human chromosome region 7q11.23 implicated in Williams Syndrome which was compared to the orthologous region of mouse chromosome 5 (DeSilva et al. Genome Research 2002, 12:3-15). This cross-species annotation of sequence allowed the identification of nine previously unreported genes, provided sequence details on 30 genes residing in the region, and revealed a number of potentially interesting conserved non-coding sequences.

In general, human and mouse sequences were found to be ˜45% similar in their intergenic regions (Shabalina et al. 2001, Trends Genet 17:373-376). Comparative sequence analysis identified gene regulatory sequences before functional studies in a second study, involving the analysis of a genomic region containing the stem cell leukaemia (SCL) gene (Gottgens et al., Nature Biotechnol. 2000, 18:181-186). Sequence comparisons showed the occurrence of frequent regions of homology within non-coding DNA. Comparisons between human and mouse identified all the previously defined SCL enhancers. Furthermore, a new neuronal enhancer close to the SCL gene was identified. These kinds of study exemplify the complexity of long-range regulatory elements and the power of comparative biology to discover and to decipher the properties of such conserved regulatory elements.

One of the main effects of computational science on molecular biology has been the development of algorithms to detect conservation between sequences. Local alignment tools, such as BLAST (Altschul et al., J. Mol. Biol. 1990, 215:403-410), were primarily developed to rapidly identify sequence similarity between a relatively short query sequence and a large sequence database. By contrast, cross-species comparisons require accurate alignment of a small number of large contiguous sequences. Whereas local alignments have been used successfully for cross-species genomic sequence comparisons, global alignments algorithms provide an overall view that specifies how two large genomic sequences fit together. Once the pieces of a large genomic interval have been aligned, smaller regions of conservation in this interval can be identified (Morgenstern et al., Batzoglou et al., Delcher et al.).

Several programs have been developed which try to retrieve information on conservation between organisms from genomic alignments, as for example, PipMaker, ADHoRe, VISTA/AVID, MUMer, WABA, Alfresco, DIALIGN and MGA. Hence, software programs have also been developed to visualize sequence alignment outputs. Visualization tools are now used for large genomic sequences, the simplest being the dotplot and the actuals being VISTA/AVID (Mayor et al., Bioinformatics 16, 1046-1047, 2000) and PipMaker (Schwartz et al., Genome Res. 10, 577-586, 2000). Both display the alignment of two or more genomes in the form of simple percentage-identity plots. VISTA (visualization tool for alignment) combines a global alignment program with a graphical tool for analyzing alignments that allows the identification of conserved coding and non-coding sequences between species. PipMaker has also been used extensively in comparative analyses. After a local sequence alignment that uses a modified version of BLAST (BLASTZ), a percentage identity plot (PIP) is generated. The PIP indicates regions of similarity based on the percentage identity of each gap-free segment of the alignment (the number of matches in the region divided by the length of the region). The VISTA algorithm requires the following as input:
(i) one or more global alignments in one of the standard formats (generated by AVID)
(ii) an annotation file for the base sequence
(iii) a set of parameters

PipMaker requires two sequence files and the optional RepeatMasker and Exon files as input. Thus, these visualization tools allow investigators to analyze sequence data from two (or more) species to visually identify conserved non-coding regions in the vicinity of genes of interest.

The previously described algorithms acts like BLAST but on a much larger scale. Their purpose is to align large genomic sequences and to possibly identify conserved regions. Besides of requiring many input information, they are not specifically designed to discover syntenic regions and will thus retrieve a high rate of false positives. PIPmaker shows the percentages using bars, with the height of each bar indicating the percentage identity in the corresponding gap-free segment. VISTA's output is a graphical plot in which the horizontal axis represents the human genomic sequence and the vertical axis indicates the percentage of identical nucleotides in a predefined interval between human and another species across the alignment.

ADHoRe (Vandepoele et al., Genome Research 2002, 12:1792-1801) algorithm is able to detect homologous regions but it requires as input a data set containing all gene products, their absolute or relative position on a genomic sequence, and their orientation.

MUMer (Delcher et al., Nucleic Acids Research 1999, 27/111:2369-2376) is a system for pairwise alignment and comparison of very large-scale DNA sequences. MUMer facilitates analysis of among others syntenic chromosomal regions. MUMer was developed for Mycoplasma studies and thus assumes that sequences are closely related. As a consequence, it can't deal with rearrangements.

WABA (Kent W J. and Zahler A. M., Genome Research 2000, 10:1115-1125) is able to recognize homologous regions at the DNA level. It has been optimized for gene-rich regions. WABA has been successfully applied to aligning the genomes of two closely related worms, Caenorhabditis briggsae and Caenorhabditis elegans. This algorithm necessitates two input files.

Alfresco (Niclas Jareborg et al., http://www.sanger.ac.uk/Software/Alfresco/) is another visualization tool that allows comparative genome sequence analysis with alignments provided by the user. The program will compare multiple sequences from putitatively homologous regions in different species. It requires two sequence files and exon information (where the exons are located and which exons in the two sequences that corresponds to each other).

DIALIGN (Morgenstern B., Bioinformatics Applications Note 2000, 16/10:948-949) constructs pairwise and multiple alignments of sequences. This method is able to identify functionally important regions even in large genomic sequences.

Similarly, MGA (Höhl M. et al., Bioinformatics 2002, 18/Suppl. 1:S312-S320) is capable of aligning three or more genomes.

LSH-ALL-PAIRS (Buhler J., Bioinformatics 2001, 17/5:419-428) is an algorithm to find ungapped local alignments in genomic sequence with up to a specified fraction of substitutions. The algorithm was used to find conserved features in several genomic sequences from human and mouse.

A few algorithms focus more specifically on the gene recognition problem by comparison of two genomic sequences: such programs are based on the hypothesis that coding DNA sequences are more conserved than non-coding sequences (intronic and intergenic). On average, human and mouse sequences are 82% identical in their coding exons, but identity drops to 50-56% in UTRs, and to 23% in introns (Jareborg et al. 1999, Genome Res. 9:815-824). Comparing two homologous genomic sequences (cross- or intra-species) should thus help to reveal conserved exons and allow the prediction of genes simultaneously on both sequences (Mathé C. et al., Nucleic Acids Research 2002, 30/19:4103-4117). Some programs like ROSETTA (Batzoglou et al., Genome Research 2000, 10:950-958) and CEM (Bafna V. and Huson D. H., Informatics Research, Celera Genomics Corp.) are more specifically designed for the comparison of closely related species. In particular, they make the hypothesis of conserved exon-intron structure in the two sequences.

ROSETTA is the first automated program that annotates human gene by using syntenic mouse genomic DNA. ROSETTA makes the further hypothesis that the corresponding exons in the two genes have roughly the same length. Hence, ROSETTA (with GLASS) were designed to accurately identify coding exons by comparison of syntenic human and mouse genomic sequences and are thus an automatic approach to exon recognition by using cross-species sequence. GLASS is designed to find short regions that match exactly and to align them.

CEM's gene finding approach simultaneously predicts complete gene structures in both human and mouse genomic sequences. It is based in part on the idea of looking for conserved protein sequences by comparing pairs of DNA sequences.

More flexibility is allowed by algorithms, which do not assume that the gene structure is conserved, as in SGP-1 (Wiehe et al., Genome Research 2001, 11:1574-1583), Pro-Gen (Novichkov et al., Bioinformatics 2001, 17/11:1011-1018) and Utopia (Blayo P., These de Doctorat, Université de Marne-la-Vallée). SGP-1 predicts protein-coding genes based on the similarity of homologous genomic sequences. SGP-1 requires a pairwise local alignment of two genomic sequences. Pro-Gen allows automated gene recognition by comparison of genomic sequences. Pro-Gen accepts as input two genomic sequences containing homologous genes. UTOPIA allows gene predictions present in two genomic sequences.

Other gene predicting softwares based on cross-species comparison include DoubleScan (Meyer I. M. and Durbin R., The Welcome Trust Sanger Institute), AgenDA (Rinner O. and Morgenstern B., In Silico Biology 2002, 2/0018), SLAM (Pachter L. et al., Journal of Computational Biology 2002, 9/2:389-399) and TWINSCAN (Korf I. et al., Bioinformatics 2001, 17/Suppl. 1:S140-S148). DoubleScan is a method that simultaneously determines the gene structures of protein-coding genes in two eukaryotic DNA sequences by using the two DNA sequences as input information. The method predicts the gene structures of the two DNA sequences, but also simultaneously retrieves the conserved subsequences within intergenic, intronic and protein coding regions.

AgenDA is a method for gene prediction that is based on long-range alignment of syntenic regions in eukaryotic genome sequences. SLAM uses a generalized pair HMM (GPHMM or dual-HMM), which can simultaneously predict a pair of ‘orthologous’ base pairs in a syntenic region. SLAM is used for modeling genes in syntenic stretches of genomic DNA from two different organisms. TWINSCAN is also a comparative-genomics-based gene-prediction system that has been designed for the analysis of high-throughput genomic (HTG) sequences.

The above algorithms designed for gene finding purposes require conserved or more specifically syntenic sequences as input. The necessity of algorithms allowing the automatic discovery of syntenic regions is therefore useful in the gene discovery field and would considerably save time in the long process of syntenic manual annotation.

The last algorithms presented here deal with comparative genomic maps. Comparative genome maps are used for predicting the location of orthologous genes, for understanding chromosome evolution and inferring phylogenetic relationships, and for examining hypotheses about the evolution of gene families and gene function in diverse organisms. The major algorithms allowing the build up of comparative genome maps are DECAL (Goldberg D. et al., Cornell University) and CONSEG (Sankoff D. et al., Journal of Computational Biology 1997, 4/4:559-565).

The DECAL algorithm is intended to reconstruct the labels on one genomic fragment in accordance to labels in another fragment. DECAL is useful in automating the process of constructing labelings based on conservation regions. DECAL doesn't deal with sequence themselves, but rather with annotations. Hence, the annotations are kind of ‘atomic’, in the sense that they cannot analyze anything inside the sequences. This approach is distinct from sequence-alignment methods, which work on a much more localized scale. For input, DECAL requires the positions of the markers of one species, as well as the location of homologs to each marker in the second species. DECAL works with regions of the genome that are already annotated in two species and orders one in terms of the other.

The CONSEG algorithm has the same objective as DECAL but the implementation is different. In particular, the CONSEG algorithm needs as input a set of conserved fragments (which might be just annotations, like in DECAL). Thus, DECAL and CONSEG assemble already recognized syntenic regions.

Tools that provide as output annotations like conservation or syntenic regions could also be useful as input for comparative maps algorithms. For example, syntenic regions could be considered as labels or markers that will be reconstructed from their positions in one species chromosomes into a second closely related species chromosomes to end up with comparative maps.

Hence, comparative genomics is a powerful tool to enhance genomic annotation, helping to locate putative genes, transcription-factor binding sites, alternative splice sites, promoters, etc., (for a review, see O'Brien et al., 1999), since functional sites in the genome tend to be more conserved during evolution than non-functional sites (Jareborg et al., 1999; Shabalina et al., 2001). However, actual identification of the correct syntenic regions for comparative analyses is a labour intensive process due to the large quantity of available information. Such a process involves the use of BLAST (Altschul et al., 1997), or another local alignment tool, of the region of interest against a set of genomic databases, followed by the identification of the sequences that contain a large number of High-scoring Segment Pairs (HSPs). While this procedure assures a high quality of the results because of frequent human intervention, it is impractical when dealing with an analysis pipeline with a large number of sequences.

In order to make studies of comparative genomics it is necessary to have at least a pair of homologous nucleotide sequences coming from different species. Starting from a single sequence of interest, the first step is to make a similarity-based search in the available databases with some standard computational tools, usually BLAST or its derivatives. The output consists of many fragments that match the query sequence to different degrees. When the sequence of interest and the database belong to the same species, the BLAST approach usually suffices to identify the “correct” matches. However, if the query sequence and the database are from different organisms, a single similarity search is not good enough because the results are not easy to interpret. Specifically, it is not easy to see if the differences between a query and a match are due to evolution, or if it is merely due to the fact that the sequences are more or less similar in one particular region, but overall are considered as not homologous, or that they may even be paralogous (i.e. a copy of the real ortholog). The only way to solve this problem is by visually inspecting a large number of matching regions to eliminate those that are actually false positives. This manual filtering, when performed by a trained person assures a high quality of the results; however, it is a very time consuming process that is not efficient to carry out in a pipeline with hundreds or thousands of sequences. Therefore, if there is a way to automate the searching and filtering process, eliminating as many false positive results as possible, the efficiency in annotating sequences of interest may increase noticeably. The present invention overcomes the actual specific limitations of the aforementioned algorithms and those of human manual interventions by providing a tool specifically designed to automatically discover syntenic regions thereby intensively reducing the time needed for the annotation process.

### SUMMARY OF THE INVENTION

In a first aspect of the invention, it provides a method for accomplishing the automatic identification of syntenic regions in one or in a plurality of given input sequence(s), comprising the steps of
(i) Detecting High-scoring Segment Pairs (HSPs) or equivalents in the target(s) genome(s) or database(s),
(ii) Filtering and construction of tiles from the HSPs or equivalents of the target(s) genome(s) or database(s),
(iii) Detecting HSPs or equivalents in the input(s) genome(s) or database(s),
(iv) Filtering and construction of tiles from the HSPs or equivalents of the input(s) genome(s) or database(s),
(v) Filtering of the tiles of the input(s) genome(s) or database(s),
(vi) Reporting the regions of the given input sequence(s) altogether with their matching tiles of the target(s) genome(s) or database(s).

In a second aspect of the invention, it provides a method of operating a computer for accomplishing the automatic identification of syntenic regions in one or in a plurality of given input sequence(s), comprising the steps of
(i) Detecting High-scoring Segment Pairs (HSPs) or equivalents in the target(s) genome(s) or database(s),
(ii) Filtering and construction of tiles from the HSPs or equivalents of the target(s) genome(s) or database(s),
(iii) Detecting HSPs or equivalents in the input(s) genome(s) or database(s),
(iv) Filtering and construction of tiles from the HSPs or equivalents of the input(s) genome(s) or database(s),
(v) Filtering of the tiles of the input(s) genome(s) or database(s),
(vi) Reporting the regions of the given input sequence(s) altogether with their matching tiles of the target(s) genome(s) or database(s).

Optionally, a last step is performed in the first or second aspect of the invention:
(vii) Using these reported matching regions of the given input sequence(s) and tiles of the target(s) genome(s) or database(s) to accomplish the practical effect of automatic identification of syntenic regions in the given input sequence(s).

In a third aspect of the invention, it provides a computer program for accomplishing the automatic identification of syntenic regions in one or in a plurality of given input sequence(s) comprising computer code means adapted to perform all steps of the first or second aspect of the invention when said program is run on a computer.

In a fourth aspect of the invention, it provides an apparatus for carrying out the method according to the first or second aspect of the invention including data input means for inserting one or a plurality of given input sequence(s) characterized in that there are provided means for carrying out the steps of the first or second aspect of the invention.

In a fifth aspect of the invention, a computer program according to the first or second aspect of the invention is embodied on a computer readable medium.

In a sixth aspect of the invention, it provides a computer readable medium having a program recorded thereon, where the program is to make the computer to carry out the method according to the first or second aspect of the invention.

In a seventh aspect of the invention, the invention provides a computer loadable product directly loadable into the internal memory of a digital computer, comprising software code portions for performing the steps of first or second aspect of the invention when said product is run on a computer.

In an eight aspect of the invention, it provides a computer program product stored on a computer usable medium, comprising computer readable program means for causing the computer to automatically detect syntenic regions in one or in a plurality of given input sequence(s) according to the first or second aspect of the invention.

### DESCRIPTION OF THE FIGURES, TABLES AND ANNEXES

FIG. 1: The figure presents the different steps involved in the first or second aspect of the OrthoFinder algorithm.

Table 1: The table indicates the mandatory and optional fields of OrthoFinder to be used in the command line.

Table 2: The table contains the performance of the algorithm at finding the corresponding syntenic regions in mouse, using human query sequences.

Table 3: The table indicates the mandatory and optional fields of OrthoPipe to be used in the command line.

Annex 1: Main custom-made perl script allowing detection of syntenic regions.

Annex 2: Custom-made perl script allowing conversion of a blast file into a set of gff entries

Annex 3: Custom-made perl script allowing sequence extraction from databases in fasta format

Annex 4: Custom-made, OrthoPipe adapted perl script of Blast2gff.

Annex 5: Custom-made, OrthoPipe adapted perl script of ConservationPlot

Annex 6: Custom-made, OrthoPipe adapted perl script of DPB.

Annex 7: Custom-made, OrthoPipe adapted perl script of MapSequence.

Annex 8: Main custom-made, OrthoPipe-adapted perl script of OrthoFinder.

Annex 9: Custom-made perl script called OrthoPipe allowing integration of OrthoFinder in a set of tools, namely Blast2gff, Mapsequence, DPB and ConservationPlot

### DETAILED DESCRIPTION OF THE INVENTION

The present invention is based on the development of an algorithm called OrthoFinder, which automatically identifies genomic syntenic regions of a given input sequence. OrthoFinder automatically locates the syntenic regions of a sequence of interest by applying a variant of the mentioned routine of blasting and filtering. Thus, the purpose of the computer program is to automate these searching and filtering procedures.

An input sequence corresponds to what is usually referred to as the query sequence. The target sequence corresponds to what is usually referred to as the subject sequence. Usually, when a local alignment tool is r=n, the query refers to the sequence that is used by the tool to find corresponding matches or subject (target) sequences in a subject or target genome or database.

In a first aspect of the invention, it provides a method for accomplishing the automatic identification of syntenic regions in one or in a plurality of given input sequence(s), comprising the steps of:
(i) Detecting High-scoring Segment Pairs (HSPs) or equivalents in the target(s) genome(s) or database(s),
(ii) Filtering and construction of tiles from the HSPs or equivalents of the target(s) genome(s) or database(s),
(iii) Detecting HSPs or equivalents in the input(s) genome(s) or database(s),
(iv) Filtering and construction of tiles from the HSPs or equivalents of the input(s) genome(s) or database(s),
(v) Filtering of the tiles of the input(s) genome(s) or database(s),
(vi) Reporting the regions of the given input sequence(s) altogether with their matching tiles of the target(s) genome(s) or database(s).

In a second aspect of the invention, it provides a method of operating a computer for accomplishing the automatic identification of syntenic regions in one or in a plurality of given input sequence(s), comprising the steps of
(i) Detecting High-scoring Segment Pairs (HSPs) or equivalents in the target(s) genome(s) or database(s),
(ii) Filtering and construction of tiles from the HSPs or equivalents of the target(s) genome(s) or database(s),
(iii) Detecting HSPs or equivalents in the input(s) genome(s) or database(s),
(iv) Filtering and construction of tiles from the HSPs or equivalents of the input(s) genome(s) or database(s),
(v) Filtering of the tiles of the input(s) genome(s) or database(s),
(vi)
(vii) Reporting the regions of the given input sequence(s) altogether with their matching tiles of the target(s) genome(s) or database(s).

Optionally, a last step is performed in the first or second aspect of the invention:
(viii) Using these reported matching regions of the given input sequence(s) and tiles of the target(s) genome(s) or database(s) to accomplish the practical effect of automatic identification of syntenic regions in the given input sequence(s).

Preferably, equivalents to HSPs are locally maximal alignments, local alignments that are considered to be relevant (having a significant score) when compared with random alignments. Equivalents can also be defined as a local similarity region or a local region of highest density of identical matches or a maximal segment pair. It is well known that significant HSPs or equivalents (i.e. having high scores or low e-values) are generally present in protein or regulatory regions (as well as introns). On the contrary, non-valuable HSPs or equivalents (i.e. having low scores or high e-values) are generally present outside the above mentioned regions and are as well usually shorter in length than high score ones.

Tiles arise by the occurrence of a collection of collinear HSPs or equivalents, i.e. having the same query and subject sequences in the same order, and correspond to regions involving the aforementioned HSPs or equivalents as well as the continuous genomic regions encompassing them. Hence, tiles refer to specific regions found in the query or subject sequence(s).

In a third aspect of the invention, it provides a computer program for accomplishing the automatic identification of syntenic regions in one or in a plurality of given input sequence(s) comprising computer code means adapted to perform all steps of the first or second aspect of the invention when said program is run on a computer.

In a fourth aspect of the invention, it provides an apparatus for carrying out the method according to the first or second aspect of the invention including data input means for inserting one or a plurality of given input sequence(s) characterized in that there are provided means for carrying out the steps of the first or second aspect of the invention.

In a fifth aspect of the invention, a computer program according to the first or second aspect of the invention is embodied on a computer readable medium.

In a sixth aspect of the invention, it provides a computer readable medium having a program recorded thereon, where the program is to make the computer to carry out the method according to the first or second aspect of the invention.

In a seventh aspect of the invention, the invention provides a computer loadable product directly loadable into the internal memory of a digital computer, comprising software code portions for performing the steps of first or second aspect of the invention when said product is run on a computer.

In an eight aspect of the invention, it provides a computer program product stored on a computer usable medium, comprising computer readable program means for causing the computer to automatically detect syntenic regions in one or in a plurality of given input sequence(s) according to the first or second aspect of the invention.

Preferably, according to the first or second aspect of the invention, the detecting of step (i) uses a local alignment tool. For example, the input sequence (for example, human sequence) is locally aligned against a genomic database of a target organism (for example, mouse).

Most preferably, such input is BLASTed using the NCBI's blastall program (NCBI suite ftp://ftp.ncbi.nih.gov/toolbox/).

Still most preferably, such input is BLASTed using the NCBI's blastall program with a word size of 16 and E-value threshold of 1e-30.

Preferably, according to the first or second aspect of the invention, in the filtering of step (ii), the raw locally aligned output obtained in step (i) is parsed to obtain the coordinates of those HSPs or their equivalents based on one or a plurality of associated criteria. The HSPs or equivalents of step (i) have for query sequence(s) the given input sequence(s) and for subject the genome(s) or database(s) of particular specie(s).

Most preferably, in the filtering of step (ii), the raw locally aligned output obtained in step (i) is parsed to obtain the coordinates of those High-scoring Segment Pairs (HSPs) or their equivalents larger than a specified length.

Still most preferably, in the filtering of step (ii), the raw locally aligned output is parsed to obtain the coordinates of those HSPs or their equivalents larger than 140 base pairs.

Still most preferably, in the filtering of step (ii), such HSPs or equivalents (i.e. larger than 140 base pairs) that overlap along the query sequence and encompass between them more than one subject (target) sequence, are kept or not based on one or a plurality of associated criteria.

Still most preferably, such HSPs or equivalents with the highest score (or lowest e-value) in any given region of overlapping of the HSPs or equivalents are kept

Then comes the identification of those genomic regions containing the remaining HSPs or equivalents.

Preferably, according to the first or second aspect of the invention, in the filtering of step (ii), whenever there is a collection of collinear HSPs or equivalents, i.e. having the same given input (query) and target (subject) sequences in the same order, the program retrieves the continuous genomic regions encompassing them, i.e. the tiles.

Preferably, according to the detecting of step (iii) of the first or second aspect of the invention, OrthoFinder tries to find the best reciprocal matches by making local alignments in the reverse direction, using the tiles from the target(s) organism(s) as queries against the genome(s) or database(s) of the organism(s) from which the original given input sequence(s) were obtained.

Most preferably, the tiles are BLASTed using the NCBI's blastall program (NCBI suite ftp://ftp.ncbi.nih.gov/toolbox/).

Still most preferably, the tiles are BLASTed using the NCBI's blastall program with a word size of 16 and E-value threshold of 1e-30.

For each locally aligned output the entire process of filtering, eliminating overlaps, and forming the tiles is repeated.

Preferably, according to the first or second aspect of the invention, in the filtering of step (iv), the raw locally aligned output obtained in step (iii) is parsed to obtain the coordinates of those High-scoring Segment Pairs (HSPs) or their equivalents based one or a plurality of associated criteria The HSPs or equivalents of step (iii) have for query sequence(s) the tiles obtained in step (ii) and for subject(s) (target) the genome(s) or database(s) of the given input sequence(s).

Most preferably, in the filtering of step (iv), the raw locally aligned output obtained in step (iii) is parsed to obtain the coordinates of those High-scoring Segment Pairs (HSPs) or their equivalents larger than a specified length.

Still most preferably, in the filtering of step (iv), the raw locally aligned output is parsed to obtain the coordinates of those HSPs or their equivalents larger than 140 base pairs.

Still most preferably, according to the filtering of step (iv) of the first or second aspect of the invention, such HSPs or equivalents (i.e. larger than 140 base pairs) that overlap along the query sequence and encompass between them more than one subject (target) sequence, are kept or not based on one or a plurality of associated criteria.

Still most preferably, the HSPs or equivalents with the highest score (or lowest e-value) in any given region of overlapping of the HSPs or equivalents are kept.

Then comes the identification of those genomic regions containing the remaining HSPs or equivalents.

Preferably, according to step (iv) of the first or second aspect of the invention, whenever there is a collection of collinear HSPs or equivalents, i.e. having the same tiles of step (ii) (new query) and new target (subject) sequences in the same order, the program retrieves the continuous genomic regions encompassing them, i.e. the tiles.

Preferably, according to the filtering of step (v) of the first or second aspect of the invention, it uses another last filter that compares the tiles of the genome(s) or database(s) of the given input sequence(s) (e.g. human tiles) against the original corresponding given input sequence(s) (e.g. human sequence).

Most preferably, it uses a local alignment tool.

Still most preferably, such tiles of the input(s) genome(s) or database(s) are BLASTed using the NCBI's blastall program (NCBI suite ftp://ftp.ncbi.nih.gov/toolbox/).

Still most preferably, only those tiles of the input(s) genome(s) or database(s) matching the original sequence with an associated probabilistic score are retained.

Still most preferably, only those tiles of the input(s) genome(s) or database(s) matching the original sequence with an E-value of 1e-30 or lower are retained.

Preferably, according to the reporting step (vi) of the first or second aspect of the invention, the program finally reports those regions of the given input sequence(s) altogether with their matching tiles of the target(s) organism(s) (i.e. genome(s) or database(s) of the organism(s) or specie(s)).

Most preferably, the invention report those regions of the given input sequence(s) altogether with their matching tiles of the target(s) organism(s) by a visualization tool or by text output

Still most preferably, the invention report those regions of the input sequence altogether with their matching tiles of the target organism by a specific format.

Most preferably, the invention reports those regions of the input sequence altogether with their matching tiles of the target organism, using as a format a set of pairs of gff entries (http://www.sanger.ac.uk/Software/formats/GFF/GFF_Spec.shtml) indicating their coordinates.

One of the advantages of the invention is that OrthoFinder is able to select two distinct types of HSPs or equivalents. Firstly, it can detect HSPs or equivalents by use of a first set of specific criteria(s), e.g., by selecting those HSPs or equivalents larger than 140 base pairs, followed by the use of a second set of specific criteria(s) in case of overlapping of such HSPs or equivalents, e.g. by selecting high-scoring HSPs or equivalents. Secondly, it can detect HSPs or equivalents that satisfy the condition of colinearity by construction of tiles. This procedure allows OrthoFinder to select the most relevant HSPs or equivalents in any sequence.

Another advantage of the invention compared with a person dedicated to this task is that human intervention will discard those HSPs having low scores by only conserving high score ones whereas OrthoFinder not necessarily. Human intervention consists of detecting and retrieving high score HSPs, thus detecting usually only the “traditional” regions. OrthoFinder, by using tiles, can consider certain HSPs having low scores on the condition of being collinear to each other. Thus, low scores HSPs will not be automatically rejected and regions of conservation are detected not only in “traditionally” conserved regions as mentioned before but also in regions located outside of these. This is of particular interest as new conservation regions can be detected compared to other manual or computer related methods.

Another advantage of the invention is that OrthoFinder uses a special filter in step (v). To avoid the possibility of the program catching paralog genes, repeats, or in general, non-syntenic regions, the program compares the input tiles (e.g. human tiles) against the original input sequence. This comparison is done by again using a local alignment tool; e.g. only those input tiles matching the original sequence with a given associated probabilistic score (for example an E-value of 1e-30 or lower) are retained. Thus, if the input tiles are in line with the criteria, orthologs are said to be detected. If not OrthoFinder rejects the tiles as not being orthologs. This step is of particular interest as it allows rejection of false positives that other programs or manual intervention retrieve. This crucial step further permits OrthoFinder's integration in a pipeline process by notably increasing the annotation's efficiency.

Still another advantage of the invention is that OrthoFinder was hence designed to do more than just detect regions of conservation. Strictly speaking, it is used to detect genomic fragments containing collinear regions of conservation. This means that between the query and target sequence there are not only conserved fragments, but there are also usually intervening non-conserved regions. The reason to this advantage lies in the way in which the tiles are constructed. Tiles are formed with the contiguous genomic regions that encompass collinear HSPs. The HSPs are the conserved regions themselves, but in-between them there are the regions that link one HSP to another, and these are the non-conserved regions that appear in the final tiles. And since the output of OrthoFinder corresponds to tiles, the output consists of regions that usually contain both conserved and non-conserved sub-regions. As a significant HSP (as mentioned before) might correspond to a gene and as it is possible to state that two collinear significant HSPs are detected, it can be deducted that the tile obtained corresponds to a syntenic region. It is therefore reasonable to state that OrthoFinder can specifically detect syntenic regions by construction of tiles. This is illustrated and confirmed by the high specificity of the invention towards the discovery of syntenic regions. By avoiding false positives, OrthoFinder is an appropriate tool for the discovery of syntenic regions as well as for its integration in a pipeline.

Evidently, the definition of syntenic regions explicitly depends on the presence of genes. If the user uses as input a region without genes, or containing only one gene, OrthoFinder will return the corresponding orthologous region, which won't be syntenic because it will contain one gene at most On the other hand, if the user feeds the program with a genomic fragment containing a few genes, then OrthoFinder will indeed return the syntenic region, because it will return the region in the other organism containing the orthologous genes in the same order. However, things can be a little more complicated than this, because of unknown (i.e. unannotated) genes present in the input sequence. If the user uses as input a region with no known genes, the output probably won't have known genes either, in which case the query and target sequences are homologous but do not seem to be syntenic. But it is possible to speculate that the genomic region used does in fact contain genes that have for the moment not been discovered and hence not yet annotated (somebody can afterwards discover that in reality there are genes in the genomic region).

Another advantage of the invention is that OrthoFinder requires only a single or a plurality of sequences or genomic fragment(s) as input (for example, from human). This is an important feature because the program can be integrated in a pipeline of lots of sequences by avoiding human intervention. Thus, no additional tools such as annotation files are requested.

Another advantage of the invention is that OrthoFinder is an efficient procedure. The automatic detection of syntenic regions is performed in such a way that it further permits its integration in a pipeline by considerably reducing the time needed for the whole process compared to the time-consuming human intervention.

It will be understood that this invention is not limited to the particular methodology, protocols, implementations and algorithms described. It is also to be understood that the terminology used herein is for the purpose of describing particular embodiments only and it is not intended that this terminology should limit the scope of the present invention. The extent of the invention is limited only by the terms of the appended claims. While the invention has been particularly shown and described with reference to a preferred embodiment thereof, it will be understood by those skilled in the art that various changes in form and details may be made therein without departing from the spirit and scope of the invention as defined by the appended claims.

Furthermore, it should be as well understood that in particular embodiments, the steps involved in this invention can be ordered differently and can be as well repeated many times without departing from the spirit and scope of the invention as defined by the appended claims.

The practice of the present invention will employ, unless otherwise indicated, conventional techniques of computer and bioinformatics skills that are within the skill of those working in the art.

Such techniques are explained fully in the literature. Examples of particularly suitable texts for consultation include the following: Attwood T. K., Parry-Smith D. J. Introduction to Bioinformatics. Addison Wesley Longman Higher Education, Essex (1999); Durbin R., Eddy S. R., Krogh A., Mitchison G. Biological sequence analysis. Probabilistic models of proteins and nucleic acids Cambridge University Press, Cambridge (1998); Wilkins M. R., Williams K. L., Appel R. D., Hochstrasser, D. F. (Editors) Proteome research: new frontiers in functional genomics. Springer Verlag Berlin Heidelberg (1997); Tisdall J. D. Beginning Perl for Bioinformatics. O'Reilly & Associates (2001); Letovsky S. I. Bioinformatics Kluwer Academic Publishers (1999); Baldi P., Brunak S. Bioinformatics The MIT Press (1998); Baxevanis A., Ouellette F. B. F. (Eds.) Bioinformatics: a practical guide to the analysis of genes and proteins John Wiley and Sons, New York (1998); Setubal J., Meidanis J. Introduction to computational molecular biology. PWS Publishing Co., Boston (1996); Schulze-Kremer S. Molecular Bioinformatics: algorithms and applications. Walter de Gruyter, Berlin -New -York (1995); Alberts B. et al. Molecular Biology of the Cell. Garland Pub (1994); Lodish H. et al. Molecular Cell Biology. W H Freeman & Co (1999); Lewin B. Genes VII. Oxford University Press (1999); Cantor C. R., Smith C. L. Genomics: The Science and Technology Behind the Human Genome Project, John Wiley & Sons, NY (1999); Bishop M. (Ed.) Guide to human genome computing. Second edition Academic Press, London (1998); Bishop M. J., Rawlings C. J. (Eds.) DNA and protein sequence analysis. A Practical approach IRL Press, Oxford (1997); Swindell S. R. (Ed.) Methods in molecular biology Vol. 70: Sequence data analysis guidebook. Humana Press, Totowa (1997); Suhai S. (Ed.) Theoretical and computational methods in genome research. Plenum Press, New York (1997); Gusfield D. Algorithms on strings, trees, and sequences. Computer science and computational biology. Cambridge University Press, Cambridge (1997); Peruski L. F. Jr., Harwood Peruski A. The Internet and the new biology: tools for genomic and molecular research. American Society for Microbiology, Washington D.C. (1997); Doolittle R. F. (Ed.) Computer methods for macromolecular sequence analysis (Methods in Enzymology, Vol. 266). Academic Press, San Diego (1996); Yap T. K., Frieder O., Martino R. L. High performance computational methods for biological sequence analysis. Kluwer Academic Publisher, Dordrecht (1996); Waterman M. S. Introduction to computational biology: maps, sequences, and genomes Chapman and Hall, London (1995); Schulze-Kremer S. Molecular Bioinformatics Walter de Gruyter (1995); Doolittle R. F Of URFs and ORFs University Science Books, Mill Valley, Calif. (1987); Adams M. D., Fields C., Venter J. C. (Eds.) Automated DNA sequencing and analysis Academic Press, London (1994); Suhai S. (Ed.) Computational methods in genome research. Plenum Press, New York (1994); Swindell S. R., Miller R. R., Myers G. S. A. (Eds.) Internet for the molecular biologist Horizon Scientific Press, Norfolk (1996); Smith D. W. (Ed.) Biocomputing. Informatics and genome projects. Academic Press, New York (1994); Griffin A. M., Griffin H. G. (Eds.) Methods in molecular biology Vol. 25: Computer analysis of sequence data, part I. Humana Press, Totowa (1994); Griffin A. M., Griffin H. G. (Eds.) Methods in molecular biology Vol. 24: Computer analysis of sequence data, part I. Humana Press, Totowa (1994); Sillince J., Sillince M. Molecular databases for protein sequences and structure studies: an introduction. Springer Verlag, Berlin (1992); Gribskov M., Devereux J. (Eds.) Sequence analysis primer Stockton Press, New York (1991); Doolittle R. F. (Ed.) Molecular Evolution: computer analysis of protein and nucleic acid sequences (Methods in Enzymology, Vol. 183). Academic Press, San Diego (1990); Waterman M. S. (Ed.) Mathematical methods for DNA sequences. CRC Press, Boca Raton (1989); Colwell R. R., Swartz D. G., McDonald M. T. (Eds.) Biomolecular data: A resource in transition. Oxford University Press, Oxford (1989); Lesk A. M. (Ed.) Computational molecular biology. Sources and methods for sequence analysis. Oxford University Press, Oxford (1988); Bishop M. J., Rawlings C. J. (Eds.) Nucleic acid and protein sequence analysis. A practical approach, IRL Press, Oxford (1987); von Heijne G.; Sequence analysis in molecular biology. Treasure trove or trivial pursuit. Academic Press, London (1987); Doolittle R. F. Of URFs and ORFs: a primer on how to analyze derived amino acid sequences. University Science Books, Mill Valley Calif. (1986); Trifonov E. N, Brendel V. GNOMIC, a dictionary of genetic codes. Balaban Publishers, Philadelphia (1986); Li W. H. Molecular Evolution (2nd Ed.) Sinauer Associates, Sunderland, Mass. (1997); “Unix Power Tools”, Jerry Peek, Tim O'Reilly & Mike Loukides, 1993. (2nd Ed.), O'Reilly Associates/Bantam, Sebastopol, Calif.

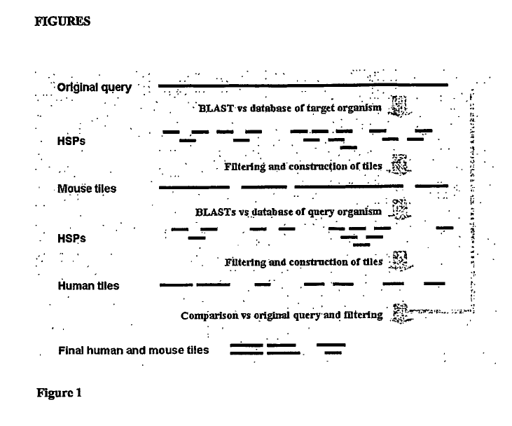

In one embodiment of the invention, OrthoFinder (see FIG. 1), implemented as a perl script, requires a single human genomic fragment as input. Such input is BLASTed against a mouse genomic database of a target organism, using the NCBI's blastall program with a word size of 16 and E-value threshold of 1e-30. These specified values can vary depending on the implementation and could be changed by the user as parameters in other embodiments. By no means they are to be considered as limiting factors and should therefore not limit the scope of the invention. Later, the raw BLAST output is parsed to obtain the coordinates of those HSPs larger than 140 base pairs. This specified length can vary depending on the implementation and could be changed by the user as a parameter in other embodiments. By no means it is to be considered as a limiting factor and should therefore not limit the scope of the invention. The next step is to keep those HSPs with the highest score in any given region of overlapping. Then comes the identification of those genomic regions containing the remaining HSPs. Whenever there is a collection of collinear HSPs, the program retrieves the tiles. Now, OrthoFinder tries to find the best reciprocal matches by making BLASTs in the reverse direction, using the tiles from the target organism as queries against the genome of the organism from which the original input sequence was obtained. For each BLAST output the entire process of filtering, eliminating overlaps, and forming the tiles is repeated. Up to now the whole process has generated triplets of regions. Each triplet consisting of a fragment of the input sequence that matches a region in the mouse genome, from which some sub-regions, in turn, match the human genome. Now comes the last filter. This comparison is done by again using BLAST; only those human tiles matching the original sequence with a low E-value (1e-30 or lower) are retained. This specified value can vary depending on the implementation and could be changed by the user as a parameter in other embodiments. By no means it is to be considered as a limiting factor and should therefore not limit the scope of the invention. Furthermore, it should be understood that other associated probabilistic methods could be used in other embodiments. By no means it is to be considered as a limiting factor and should therefore not limit the scope of the invention. Finally, the program reports those regions of the input sequence altogether with their matching tiles of the target organism, using as a format a set of pairs of gff entries (http://www.sanger. ac. uk/Software/formats/GFF/GFF_Spec.shtml) indicating their coordinates. It should be understood that other formats could be used in other embodiments. By no means this format is to be considered as a limiting factor and should therefore not limit the scope of the invention.

In a preferred embodiment of the method, the OrthoFinder perl script makes use of external programs from the NCBI suite (ftp://ftp.ncbi.nih.gov/toolbox/), called blastall and formatdb (formatdb is the program that generates index files for BLAST processing), and one form the EMBOSS package (http:/www.hgmp.mrc.ac.uk/Software/EMBOSS/), called seqret (a program that reads and writes (returns) a sequence). It should be understood that other external programs could be used in other embodiments. By no means these external programs are to be considered as a limiting factor and should therefore not limit the scope of the invention.

In another preferred embodiment, it furthermore needs two custom-made perl modules for parsing and sequence retrieval. While the scripts are adapted to an in-house computing environment, it is straightforward to modify them to suit other environments. In a further preferred embodiment of the invention, values for the different parameters used by OrthoFinder are optimized to yield high specificity results when using human sequence as input, and mouse as the target species. These specified values can vary depending on the implementation and could be changed by the user as parameter(s) in other embodiments. By no means they are to be considered as a limiting factor and should therefore not limit the scope of the invention.

In still another preferred embodiment, OrthoFinder uses the mandatory and optional fields as indicated in table 1 for the command line. The optional fields leave the opportunity for the user to optimize his results, resulting in new default values specific for a particular pair of organisms in a manner described in the first embodiment of the invention, which will therefore result in an increased consistency of the results. Furthermore, in this embodiment, the user can choose the query and target organisms from which the syntenic regions will be determined. In other embodiments of the invention, it is possible to fix the query and target organisms (e.g. in one embodiment the query organism is set to human and the target organism is set to mouse, in another embodiment the query organism is set to human and the target organism is set to rat), leaving no choice for the user, who is left with the sole “input” as mandatory field. If a multitude of embodiments are created in that manner (i.e. many embodiments where each have a unique pair of selected organisms), the user will have the choice of selecting particular pairs of organisms. More conveniently, the query and target organisms have default settings (for example, the query organism is set to human and the target organism is set to mouse), which can be changed by the user. After the selection by the user of the organisms by an optional field, the default settings will be changed accordingly, leaving the user with one mandatory field only (i.e. the input field) for consecutive uses.

TABLE 1

| | Description |
|---|---|
| Mandatory fields | |
| input | the file that contains your sequence |
| | of interest in fasta format |
| query_organism_database | a blastable database containing the genome |
| | of the same species as your input sequence |
| target_organism_database | a blastable database with the genome |
| | of the organism where you want to find |
| | an ortholog |
| Optional fields | |
| word | the “word” parameter for the blasts, default |
| | is 16 |
| blast_threshold | value to use in the “−e” parameter of blast, |
| | default is 1e−30 |
| processors | number of processors to use for the blast |
| | default is 30 |
| size_threshold | HSPs below this size will be ignored |
| | while parsing the blast output, default is 140 |
| output | destination of the results, default is |
| | STDOUT |

In another embodiment, the user, using one command line only, is able to retrieve the syntenic regions of one input in many organisms, which are either selected by the user or fixed by default settings. The user will end up with a semi-2D matrix displaying the syntenic regions for all the pair of organisms. The matrix can be populated by the following method: the first input is used against all the other different organisms for the discovery of syntenic regions in a parallel (or simultaneous) manner. For example, if the input's organism is human, this human input will be used each time by the program to discover the syntenic regions in the other organisms. If the other organisms are for example mouse, rat and rabbit, the program will perform the following procedure: it will, in a parallel manner, using the human input only, discover the syntenic regions in the following pairs of organisms respectively: human-mouse, human-rat and human-rabbit. This procedure enables the program to automatically populate the 2D-matrix for the following pairs of organisms: mouse-rat, mouse-rabbit and rat-rabbit

In a further embodiment, OrthoFinder can be combined to a set of programs or pipeline. The invention therefore also encompasses the integration of OrthoFinder with a set of tools. This set of programs can use the same mandatory and optional fields as indicated in table 1 for the command line, with the addition of an extra optional flag field permitting the user to indicate if the input is cDNA. In this way, the kind of information retrieved by the user is expanded. Even though OrthoFinder is combined to a set of programs, each of them can still be run as stand-alone and not only as an integrated whole. The user can therefore focus on one kind of analysis only or make a whole process of comparative genomics using the whole bench of programs typing only one command line.

For all algorithms aimed at analyzing sequences, it is possible to measure their performance by measuring their specificity (rate of true positives) and sensitivity (rate of overall detection). The implemented algorithm is capable of finding syntenic regions with high specificity; namely, 90% or more depending on the sequence. Hence, the optimization of the algorithm allows it to return matching sequences with high specificity, i.e. with a low rate of false positives, making it suitable for its integration in genomic annotation pipelines. True, human intervention is always needed in any kind of annotation, but by importantly reducing the time required to eliminate most of the false positives the speed of analysis can be increased noticeably.

It should be understood that other programming languages, specific values, implementations, associated or external programs, algorithms, formats, interfaces, outputs could be used in other embodiments in order to perform the invention. By no means these are to be considered as limiting factors and should therefore not limit the scope of the invention.

### EXAMPLES

### Example 1

To obtain optimized values for the different parameters used by OrthoFinder in order to yield high specificity results when using human sequence as input, and mouse as the target species, two sets of training sequences were used. The first was a set of 77 human-mouse ortholog genes (Jareborg et al., 1999; http://www.sanger.ac.uk/Software/Alfresco/mmhs.shtml). These are sequences with a high coding to non-coding ratio. However, the algorithm was also trained with genomic fragments with a larger proportion of non-coding regions. For this purpose, the complete set of RefSeq (Pruitt and Maglott, 2001) entries from human chromosome 19 was used for which there are annotated mouse gene orthologs. The publicly available annotations were retrieved and compiled into a database to use it as second training set, available as supplementary material (http:/www.ncbi.nlm.nih.gov/LocusLink/refseq.html). As test sets two other databases were used, one containing genomic sequences spanning one gene each (Batzoglou et al., 2000; http://crosssopecies.lcs.mit.edu/), and the other was the complete RefSeq set of entries of human chromosome 18 that contained at least one annotation for mouse synteny (data obtained from http://www.ensembl.org/).

For comparative purposes, the performance of Godzilla, the Berkeley genome pipeline (http://pipeline.lbl.gov/) was also analyzed. Because of size restriction in the submitted sequences, comparative study for only two of the databases were performed.

Table 1 contains the performance of the algorithm at finding the corresponding syntenic regions in mouse, using human query sequences. As it can be seen, the OrthoFinder algorithm shows a high value of specificity. This characteristic of the algorithm makes it very useful when dealing with a pipeline of hundreds or thousands of sequences, saving time in the process of BLASTing, selecting sequences and filtering, in order to find regions of synteny.

The comparative analysis showed that the algorithm is clearly more specific than Berkeley's. Godzilla is more sensitive, meaning that it detects more regions, but its rate of false positives is extremely high, thus not being good enough for its integration in a pipeline.

The Blast results correspond to a typical manual process of syntenic detection. Blast was run on each of the Batzoglou sequences. The highest ranked HSP was then chosen by human intervention, and looked if such HSP belonged to the correct region of orthology. Hence, this procedure of taking the highest-ranked HSP is what a biologist most often does. Hence these results give a comparison of manual intervention with OrthoFinder. Manual intervention, as Godzilla, is more sensitive, but its rate of false positives is higher than OrthoFinder. These results also further indicate that OrthoFinder is an appropriate tool for the discovery of syntenic regions as well as for its integration in a pipeline.

TABLE 2
COMPARATIVE PERFORMANCE OF ORTHOFINDER

| | Sensitivity ^{a} | Specificity ^{b} |
|---|---|---|
| OrthoFinder on Jareborg (training) dataset | 0.8000 | 0.9411 |
| OrthoFinder on Chr19 (training) dataset | 0.8421 | 0.9142 |
| OrthoFinder on Batzoglou dataset | 0.6410 | 0.9615 |
| OrthoFinder on Chr18 dataset | 0.8529 | 0.9062 |
| Godzilla on Jareborg dataset | 1.0000 | 0.5797 |
| Godzilla on Batzoglou dataset | 0.9145 | 0.4736 |
| BLAST on Batzoglou dataset | 0.8031 | 0.8160 |

^{a} Defined as the number of correctly predicted syntenic regions divided by the number of annotated syntenic regions.
^{b} Defined as the number of correctly predicted syntenic regions divided by the total number of predicted syntenic regions.

### Example 2

OrthoFinder has been incorporated to a set or pipeline of tools useful in comparative genomics. Instead of being only one program, OrthoFinder is now part of a suite of programs called OrthoPipe. While the algorithm behind OrthoFinder remains the same, the kind of information received by the user has been expanded. OrthoPipe is made of the six following programs:
Blast2gff, converts the raw blast output into gff format
MapSequence, maps a cDNA to the genome or genomic DNA to another assembly
OrthoFinder, finds the syntenic region of a query sequence in the genome of another species
DPB, makes pairwise global alignments of nucleotides
ConservationPlot, makes a graph of global alignments
OrthoPipe, a program that integrates the above-mentioned 5 into one

In OrthoPipe, the programs can be run as stand-alone or as an integrated whole, so the user can focus on one kind of analysis or make the whole process of comparative genomics typing only one command line. OrthoPipe is therefore a script that integrates the previous five programs into a single one (see Annexes for the scripts). It takes as input a cDNA or a DNA sequence, altogether with the path to the genomic databases of the query organism (for example human), and target organism (for example mouse). Table 2 indicates the mandatory and optional field.

TABLE 3

| | Description |
|---|---|
| Mandatory fields | |
| input | query sequence in fasta format |
| query_organism_database | genome database of the query organism |
| target_organism_database | genome database of the target organism |
| Optional fields: | |
| cdna | flag, use it if your input sequence is cDNA |

The output is the set of four/five files with the following extensions:

| | | |
|---|---|---|
| i) | mapped.gff | generated by MapSequence, if the |
| | | input was cDNA |
| ii) | syntenic.gff | generated by OrthoFinder |
| iii) | alignment_1.aln | generated by DPB |
| iv) | alignment_1.pff | generated by DPS |
| v) | conservation_plot_1.png | generated by ConservationPlot |

This program works as follows: if the -cdna is indicated; it maps the sequence to the genome of the query organism. This genomic sequence is then fed into OrthoFinder. By contrast, if the input is DNA, it is directed to OrthoFinder straightaway. OrthoFinder obtains the syntenic region in the target organism. Then, DPB aligns the genomic sequences of the query and target organisms, and the Clustal output is redirected to ConservationPlot. It is important to note that OrthoPipe uses the default parameters of the other programs. Hence, if the parameters need to be changed (to enhance the results for some particular organisms, for example), then it will be necessary to either i) run the scripts one by one indicating the new parametric values, or ii) modify the default values in an initialize subroutine of the relevant scripts, and then run OrthoPipe.

1-46. (canceled)

47. A method for accomplishing the automatic identification of syntenic regions in one or in a plurality of given input sequence(s), comprising the steps of:
i) detecting High-scoring Segment Pairs (HSPs) or equivalents in target(s) genome(s) or database(s);
ii) filtering and construction of tiles from said HSPs or equivalents of said target(s) genome(s) or database(s);
iii) detecting HSPs or equivalents in input(s) genome(s) or database(s);
iv) filtering and construction of tiles from said HSPs or equivalents of said input(s) genome(s) or database(s);
v) filtering of said tiles of said input(s) genome(s) or database(s); and
vi) reporting regions of said given input sequence(s) altogether with their matching tiles of said target(s) genome(s) or database(s).

48. The method according to claim 47, further comprising using said reported matching regions of said given input sequence(s) and tiles of said target(s) genome(s) or database(s) to automatically identify syntenic regions in said given input sequence(s).

49. The method according to claim 47, wherein said equivalents encompass locally maximal alignments, or local alignments which are considered to be relevant when compared with random alignments, or local similarity regions, or local regions of highest density of identical matches, or maximal segment pairs.

50. The method according to claim 47, wherein said detecting of said step (i) uses a local alignment tool.

51. The method according to claim 50, wherein said detecting of said step (i) uses the NCBI's blastall program.

52. The method according to claim 51, wherein said detecting of step (i) uses the NCBI's blastall program with a word size of 16 and E-value threshold of 1e-30.

53. The method according to claim 52, wherein said High-scoring Segment Pairs (HSPs) or said equivalents of said step (i) are subject to said filtering of said step (ii) by one or a plurality of associated criteria.

54. The method according to claim 53, wherein said criteria is a determined length of said High-scoring Segment Pairs (HSPs) or said equivalents of said step (i).

55. The method according to claim 54, wherein said determined length is larger than 140 base pairs.

56. The method according to claim 53, wherein overlapping said High-scoring Segment Pairs (HSPs) along said given input sequence or plurality of given input sequences and encompassing between them more than one target sequence, are subject to said filtering of said step (ii), by one or a plurality of associated criteria.

57. The method according to claim 56, wherein said criteria is the keeping of said overlapping HSPs or said equivalents having the highest score or lowest e-value in any given region of said overlapping High-scoring Segment Pairs (HSPs) or said equivalents.

58. The method according to claim 47, wherein said tiles of said step (ii) correspond to the continuous genomic regions encompassing a collection of collinear said HSPs or said equivalents of said step (i).

59. The method according to claim 47, wherein said detecting of said step (iii) uses a local alignment tool.

60. The method according to claim 59, wherein said detecting of said step (iii) uses the NCBI's blastall program.

61. The method according to claim 60, wherein said detecting of said step (iii) uses the NCBI's blastall program with a word size of 16 and E-value threshold of 1e-30.

62. The method according to claim 47, wherein said High-scoring Segment Pairs (HSPs) or said equivalents of said step (iii) are subject to said filtering of said step (iv) by one or a plurality of associated criteria.

63. The method according to claim 47, wherein said criteria is a determined length of the said High-scoring Segment Pairs (HSPs) or said equivalents of said step (iii).

64. The method according to claim 63, wherein said determined length is larger than 140 base pairs.

65. The method according to claim 62, wherein overlapping said High-scoring Segment Pairs (HSPs) along said given input sequence or plurality of given input sequences and encompassing between them more than one target sequence, are subject to said filtering of said step (iv), by one or a plurality of associated criteria.

66. The method according to claim 65, wherein said criteria is the keeping of said overlapping HSPs or said equivalents having the highest score or lowest e-value in any given region of said overlapping High-scoring Segment Pairs (HSPs) or said equivalents.

67. The method according to claim 47, wherein said tiles of said step (iv) correspond to the continuous genomic regions encompassing a collection of collinear said HSPs or equivalents of said step (iii).

68. The method according to claim 47, wherein said filtering of said tiles of said step (v) is performed by comparison of said tiles of said step (v) against the original said given input sequence or plurality of given input sequences.

69. The method according to claim 68, wherein said comparison is performed by a local alignment tool.

70. The method according to claim 69, wherein said local alignment tool is the NCBI's blastall program.

71. The method according to claim 70, wherein said filtering of said tiles of said step (v) is performed by an associated probabilistic score.

72. The method according to claim 71, wherein said associated probabilistic score is an E-value of 1e-30.

73. The method according to claim 47, wherein said reporting of said step (vi) is done using a visualization tool or a text output.

74. The method according to claim 73, wherein said text output is a specific format.

75. The method according to claim 74, wherein said format is a set of pairs of gff entries.

76. The method according to claim 47, wherein said method is integrated in a pipeline.

77. The method according to claim 76, wherein said integration in a pipeline is done by OrthoPipe, and wherein the pipeline of tools consists of Blast2gff, MapSequence, OrthoFinder, DPB and ConservationPlot.

78. The method according to claim 47, wherein said method detects syntenic regions based on non-valuable or low-score or high e-values HSPs or equivalents.

79. The method according to claim 47, wherein said method requires only one or a plurality of given input sequences in order to accomplish the practical effect of automatic detection of syntenic regions in a given input sequence or in a plurality of given input sequences.

80. The method according to claim 47, wherein said method uses external programs from the NCBI suite called blastall and formatdb, and one external program from the EMBOSS package called seqret.

81. The method according to claim 47, wherein a given input sequence is used for the detection of syntenic regions in a plurality of target organisms.

82. The method according to claim 81, wherein said detection allows automatic determination of the resulting pairs of syntenic regions for all and each pairs of organisms.

83. The method according to claim 82, wherein the reporting of results is done by means of a 2D matrix.

84. The method according to claim 81, wherein a plurality of input sequences are used.

85. The method according to claim 84, wherein the reporting of results is done by means of a plurality of 2D matrices, each corresponding to one given input sequence.

86. The method according to claim 47, wherein said method is performed via the operation of a computer.

87. A computer program for the automatic identification of syntenic regions in one or in a plurality of given input sequence(s) comprising computer code means adapted to perform the steps according to claim 47 when said program is run on a computer.

88. The computer program according to claim 87, wherein said computer program is recorded on a computer readable medium.

89. A computer loadable product directly loadable into the internal memory of a digital computer, comprising software code portions for performing the method of claim 47 when said product is run on a computer.

90. An apparatus for performing the method of claim 47 that comprises data input means for inserting one or a plurality of given input sequence(s) and means for carrying out the method.
